(19) United States
(12) Patent Application Publication
(10) Pub. No.: US 2009/0264528 A1
(43) Pub. Date: Oct. 22, 2009

(54) SERINE PALMITOYLCOA TRANSFERASE (SPT) INHIBITION BY MYRIOCIN OR GENETIC DEFICIENCY DECREASES CHOLESTEROL ABSORPTION

(75) Inventors: Xian-Cheng Jiang, Fort Lee, NJ (US); Zhiqiang Li, Brooklyn, NY (US)

Correspondence Address: HOFFMANN & BARON, LLP, 6900 JERICHO TURNPIKE, SYOSSET, NY, 11791, US

(73) Assignee: The Research Foundation of State University of New York, Albany, NY (US)

(21) Appl. No.: 12/425,818

(22) Filed: Apr. 17, 2009

Related U.S. Application Data
(60) Provisional application No. 61/046,215, filed on Apr. 18, 2008.

(51) Int. Cl. A61K 31/201; G01N 33/68; G01N 33/92; C12Q 1/02

(52) U.S. Cl. 514/560; 436/86; 436/71; 435/29

(57) ABSTRACT

A first aspect of the invention provides a method of screening cholesterol absorption inhibitors, including: administering to a mammal a biologically effective amount of a candidate SPT inhibitor; and determining whether an amount at least one cholesterol absorption indicator protein in the intestine has changed after the administering step.

### CROSS REFERENCE TO RELATED APPLICATIONS

This application claims the benefit of and priority to U.S. Application No. 61/046,215, filed on Apr. 18, 2008, the contents of which are incorporated herein by reference in its entirety.

### FUNDING STATEMENT

This invention was made with government support under contract identifier HL-69817 and HL-64735 from the National Institute of Heath and by contract identifier Grant-in-Aid 0755922T from the American Heart Association. The government has certain rights to the invention.

### FIELD OF THE INVENTION

The invention relates to a screening methods for determining whether a candidate agent is an SPT inhibiting agent. Specifically, the invention relates to screening SPT inhibitors to biochemically modify the small intestine to prevent cholesterol absorption.

### RELATED ART

There are currently many cholesterol drugs constantly being developed and those currently on the market. However, some of these drugs change the pathology of the body, particularly at the site of absorption. Other drugs, like myriocin, have cholesterol absorption inhibiting capabilities, but have a toxicity associated with its administration. Thus, there currently exists a need in the art for a method of screening potential SPT inhibitors to determine successful candidates that inhibit SPT and thus, inhibit cholesterol absorption.

### SUMMARY OF THE INVENTION

A first aspect of the invention provides a method of screening cholesterol absorption inhibitors, including: administering to a mammal a biologically effective amount of a candidate SPT inhibitor; and determining whether an amount of at least one cholesterol absorption indicator protein in the intestine has changed after the administering step.

A second aspect of the invention provides a method of biochemically reducing cholesterol absorption, including administering to the mammal a biologically effective amount of an SPT inhibitor that reduces at least one of an NPC1L1 level and an ABCA1 level, and increases an ABCG5 level.

These and other features of the invention will be better understood through a study of the following detailed description and accompanying drawings.

### BRIEF DESCRIPTION OF DRAWINGS

FIGS. 1A-1C depict three graphical representations of SPT deficiency decreased SPT activity in mouse small intestine. SPT activity of small intestine homogenate was measured with [³H]serine and palmitoyl-coenzyme A as substrates. TLC was performed to separate the product, 3-keto-dihydrosphingosine (KDS, Inset). KDS band was scanned. In FIG. 1A, wildtype (WT) mice fed chow diet or chow diet plus myriocin. In FIG. 1B, apoE KO (knockout)

mice fed chow diet or chow diet plus myriocin. In FIG. 1C, WT and Sptlc1^{+/-} mice.

Values are mean±SD (n=5, P<0.01).

FIGS. 2A-D depict four images of the small intestine surface at various magnifications which illustrates that SPT deficiency did not change morphology of mouse small intestine. The small intestine was dissected out and put into 4% paraformaldehyde for fixation overnight. The tissue was then sliced (10 micrometer thick). Each slice was deparaffinized and stained with hematoxylin and eosin. The morphology was checked by microscope. WT mice fed chow diet (FIGS. 2A, 2B) or chow diet plus myriocin (FIGS. 2C, 2D). FIGS. 2A and 2C are at 10× magnification, while FIGS. 2B and 2D are at 40× magnification.

FIGS. 3A-D depict four images of the small intestine surface at various magnifications which illustrates that SPT deficiency did not change morphology of mouse small intestine. The small intestine was dissected out and put into 4% paraformaldehyde for fixation overnight. The tissue was then sliced (10 micrometer thick). Each slice was deparaffinized and stained with hematoxylin and eosin. The morphology was checked by microscope. apoE KO mice fed chow diet (FIGS. 3A, 3B) or chow diet plus myriocin (FIGS. 3C, 3D). FIGS. 3A and 3C are at 10× magnification, while FIGS. 3B and 3D are at 40× magnification.

FIGS. 4A-D depict four images of the small intestine surface at various magnifications which illustrates that SPT deficiency did not change morphology of mouse small intestine. The small intestine was dissected out and put into 4% paraformaldehyde for fixation overnight. The tissue was then sliced (10 micrometer thick). Each slice was deparaffinized and stained with hematoxylin and eosin. The morphology was checked by microscope. depicts WT mice (FIGS. 4A, 4B) and Sptlc1^{+/-} mice (FIGS. 4C, 4D). FIGS. 4A and 4C are at 10× magnification, while FIGS. 4B and 4D are at 40× magnification.

FIGS. 5A-I depict several graphs that illustrate that SPT deficient mice absorbed less cholesterol. Mice (n=6, 10-12 weeks old) were fasted for 5 hours and gavaged with 0.1 microCi [¹⁴C]cholesterol and 0.2 microCi [³H]sitostanol with 0.5 mg unlabeled cholesterol dissolved in 15 microl of olive oil. Feces were collected after 24 hours and lipids were extracted for counting (FIGS. 5A, 5D, and 5G, cholesterol absorption, WT vs WT/myriocin; apoE KO vs apoE KO/myriocin; WT vs Sptlc1^{+/-}, respectively). Blood was collected during the 24 hour period (Panels B, E, and H, [¹⁴C]cholesterol in blood, WT vs WT/myriocin; apoE KO vs apoE KO/myriocin; WT vs Sptlc1^{+/-}, respectively). To test specificity, the mice were gavaged with 0.2 microCi [³H]triolein together with 0.5 mg/ml cold cholesterol. Blood was collected during the 24 hour period (FIGS. 5C, 5F, and 5I, [³H] glycerolipids in blood, WT versus WT/myriocin; apoE KO versus apoE KO/myriocin; WT versus Sptlc1^{+/-}, respectively). Value is mean±SD. *P<0.01.

FIGS. 6A-D depict several graphs that illustrate that SPT deficient enterocytes absorb less cholesterol than controls. Enterocytes were isolated from mice (n=4, 12-14 weeks old) and resuspended in 4 ml of DMEM containing 0.05 microCi/ml of either [¹⁴C]cholesterol (FIGS. 6A and 6C) or [³H]oleic acid (FIGS. 6B and 6D). Enterocytes (100 microl) were collected at 5, 10, and 20 minutes, washed twice with DMEM, and cellular radioactivity was determined. The amounts of lipids were normalized to protein and plotted against time. FIGS. 6A and 6B, WT vs WT/myriocin; FIGS. 6C and 6D, WT vs Sptlc1^{+/-}, Values are mean±SD. *P<0.01.

FIGS. 7A-B depict several graphs that illustrate that SPT deficiency decreased SM levels on the apical surface of mouse small intestine. Small intestines were turned inside-out and cut into 0.5 cm of segments from jejunum. The segments were then bathed in 0.5 ml of oxygenated DMEM containing 5% glutamine with 5 microg/ml lysenin or without it as control, at 37 degrees C. for 30 minutes. Then 0.05 ml of WST-1 cell proliferation reagent were added and continuously incubated at 37 degrees C. for 15 minutes. After incubation, solutions were transferred to an Eppendorf tube and quick-spun at maximum speed to pellet cell debris. Supernatants were read OD at 450 nm, a reading for viable cells. Background reading was the WST-1 reagent with no cell treatment. FIG. 7A, WT vs WT/myriocin; FIG. 7B, WT vs Sptlc1^{+/-}. Value is mean±SD (n=4). *P<0.01.

FIG. 8 depicts that SPT deficiency decreased NPC1L1 and ABCA1, and increased ABCG5 in mouse small intestine. Tissue homogenates were prepared as described in Methods, and equal amounts of homogenates from each mouse were pooled in each group (n=5). A total of 50 microg pooled sample from each group was immunoblotted with antibodies against NPC1L1, ABCA1, and ABCG5. Immunoblots of beta (β)-actin were used as a loading control. These results are representative of three independent experiments.

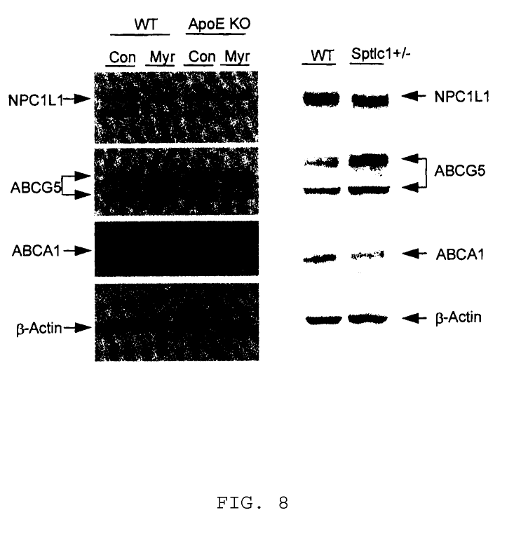

FIGS. 9A-F depict that Mouse small intestine and liver NPC1L1, ABCG5/G8, and ABCA1 mRNA measurements. NPC1L1, ABCG5/G8, and ABCA1 mRNA levels were measured by real-time PCR as described in “Methods and Materials.” FIG. 9A, jejunum from WT mice with or without myriocin treatment; FIG. 9B, jejunum from apoE KO mice with or without myriocin treatment; FIG. 9C, jejunum from WT and Sptlc1^{+/-} mice; FIG. 9D, liver from WT mice with or without myriocin treatment; FIG. 9E, liver from apoE KO mice with or without myriocin treatment; FIG. 9F, liver from WT and Sptlc1^{+/-} mice. Values are mean±SD, n=5. *P<0.01.

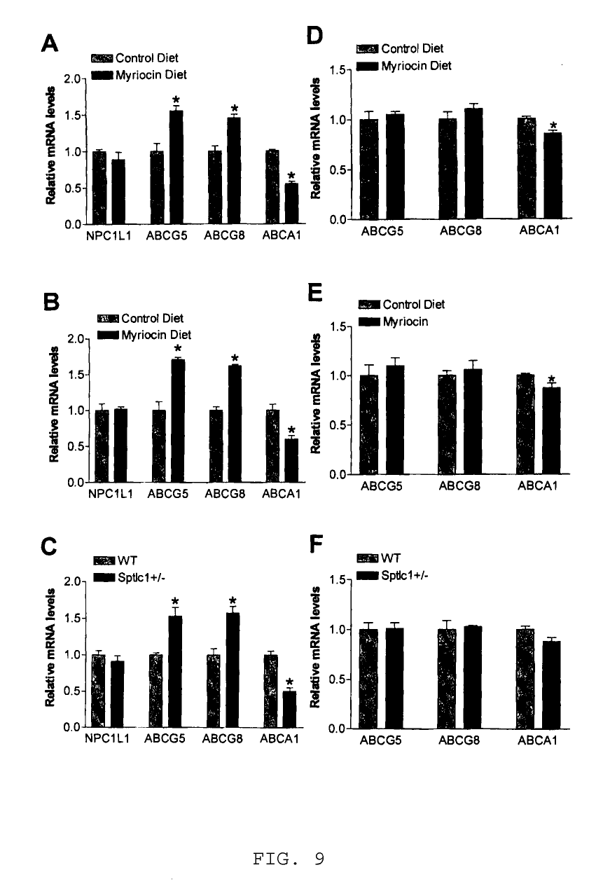

### DETAILED DESCRIPTION OF THE INVENTION

Lowering plasma cholesterol is important because of the cardiovascular and metabolic disorders associated with high levels of it. Some 30% of this cholesterol is derived by intestinal absorption. It has been estimated that a 60% reduction in plasma cholesterol levels could be achieved by total inhibition of cholesterol absorption. Absorption is a multi-step process in which cholesterol is micellized by bile acids and phospholipids in the intestinal lumen, taken up by the enterocytes, assembled into lipoproteins, and transported to the lymph and the circulation. “Enterocytes”, as referenced herein, may refer to intestinal absorptive cells and are simple columnar epithelial cells found in the small intestine and colon.

Serine palmitoylCoA transferase (SPT) is the key enzyme for the biosynthesis of sphingolipids. Upon studying the cholesterol absorption in myriocin-treated WT or apoE KO animals, the present inventors made the unexpected discovery that, after myriocin treatment, the mice absorbed significantly less cholesterol than controls, with no observable pathological changes in the small intestine. More important, the present inventors discovered unexpectedly that heterozygous Sptlc1 (a subunit of SPT, serine palmitoylCoA transferase long chain base subunit 1) KO mice also absorbed significantly less cholesterol than controls. To understand the mechanism, protein levels of Niemann-Pick C1-like 1 (NPC1L1), ABCG5, and ABCA1, three key factors involved in intestinal cholesterol absorption, were measured.

Surprisingly, the present inventors found that NPC1L1 and ABCA1 were decreased, whereas ABCG5 was increased in the SPT-deficient small intestine. Also, sphingomyelin (SM) levels on the apical were measured and it was found that the SM levels were significantly decreased in SPT deficient mice, compared with controls.

Therefore, as the present invention provides, SPT deficiency may desirably reduce intestinal cholesterol absorption by altering NPC1L1 and ABCG5 protein levels in the apical membranes of enterocytes through lowering apical membrane SM levels. Also, the same may apply to the protein ABCA1 which locates on basal membrane of enterocytes. Thus, this mechanism may be used to screen for SPT inhibitors and cholesterol absorption inhibitors. Further, the manipulation of SPT activity can provide a novel alternative treatment for dyslipidemia and other related overproduction, deficiencies, and disorders.

Accumulating evidence indicates that Niemann-Pick C1-like 1 (NPC1L1) protein plays a key role in the influx of cholesterol into the enterocytes. After uptake, some of the cholesterol is believed capable of secreting back into the intestinal lumen by means of the heterodimeric ATP-binding cassette transporters G5 and G8 (ABCG5/G8). A majority of the cholesterol taken up by the enterocytes is transported to the plasma. It has been shown that this process may involve at least two pathways: apolipoprotein (apo) B-dependent, and apoB-independent. The apoB-dependent pathway requires apoB and microsomal triglyceride transfer protein (MTP) activity. Apo AI and ABCA1 have been shown to play a role in the apoB-independent or the HDL pathway.

Located in the endoplasmic reticulum (ER) membranes, SPT is the rate-limiting enzyme in the biosynthesis of sphingolipids, including sphingomyelin (SM). Mammalian SPT contains two subunits, Sptlc1 and Sptlc2, encoding 53- and 63-kDa proteins, respectively.

The interaction of SM, cholesterol, and glycosphingolipids drives the formation of plasma membrane rafts. These lipid rafts, formed in the Golgi apparatus, are targeted to the plasma membranes, where they are thought to exist as islands within the sea of bulk membranes. Despite disagreement as to their content, the rafts are considered in most reports to comprise approximately 3500 lipid molecules and 30 proteins. As much as 65% of total cellular SM is located in these rafts.

The apical membrane of an intestinal cell is enriched in SM and cholesterol, indicative of the presence of lipid rafts. It has been proposed that lipid rafts in the enterocyte apical membranes play an important role in cholesterol absorption and trafficking. Both NPC1L1 and ABCG5/G8 are expressed in the small intestine and localized at the enterocyte apical surface.

The present invention is aimed at altering the SM levels on this surface in order to change NPC1L1 and ABCG5/G8 levels, thus influencing cholesterol absorption. Further, this knowledge may be used, as in the present invention, as a way to test candidate drugs which inhibit or otherwise decrease shingomyelin presence. Alternatively, this knowledge may be used to test candidate drugs which can inhibit cholesterol absorption from the small intestine of an animal or mammal subject.

The present inventors utilized a pharmacological approach (myriocin treatment) and a genetic one (Sptlc1 gene knockout) to investigate the effect of SPT deficiency on cholesterol absorption. The present inventors tested the hypothesis that intestine-specific SPT deficiency may alter NPC1L1 and ABCG5/G8 protein levels by directly influencing the apical surface structure of the enterocytes, thereby reducing cholesterol absorption.

There is a need for a method to effectively screen SPT inhibitor candidates and cholesterol absorption inhibitor candidates, where the candidates are chemical compounds and/or medicaments, in order to determine whether the compounds inhibit sphingomyelin formation in and/or cholesterol absorption in the small intestine.

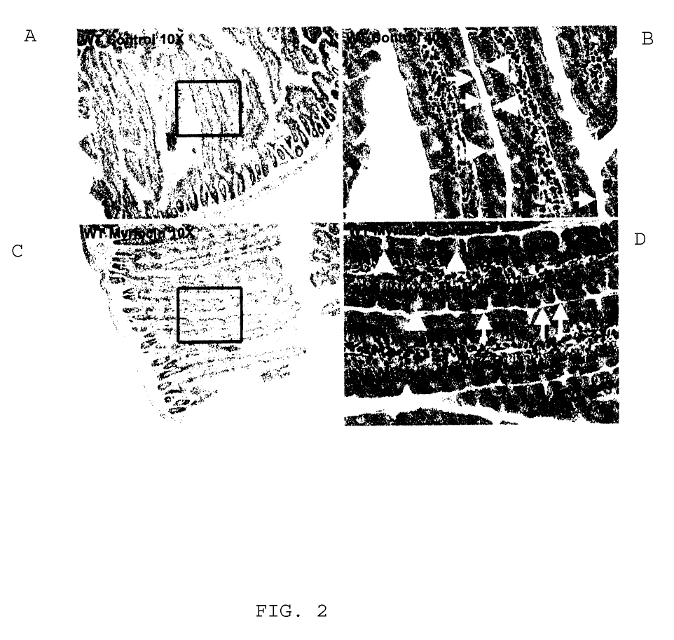

To investigate possible gastrointestinal toxicity, the morphology of the small intestine after myriocin treatment was specifically examined and no pathological changes were found in the tissue. As shown in FIGS. 2 through 4, all specimens had intact villi, epithelia, enterocytes, goblet cells, and brush borders. This was likewise true for the small intestine specimens obtained from Sptlc1^{+/-} mice. Thus, myriocin treatment and Sptlc1 gene deficiency cause biochemical rather than morphological changes in the small intestine.

SPT deficiency reduced cholesterol absorption, which would tend to explain why only apoE KO mice had less cholesterol in the circulation. ApoE-mediated cholesterol clearance is a major pathway for removing chylomicron and remnant cholesterol from the circulation.(26) Since apoE KO mice have a defect in this removal pathway,(27) the decrease in cholesterol absorption would be reflected in plasma cholesterol levels (Table 1). On the other hand, WT and Sptlc1^{+/-} animals have a normal apoE-mediated cholesterol clearance pathway and do not accumulate chylomicron remnants, so it is not surprising that the defect in cholesterol absorption is not reflected in plasma cholesterol levels (Table 1).

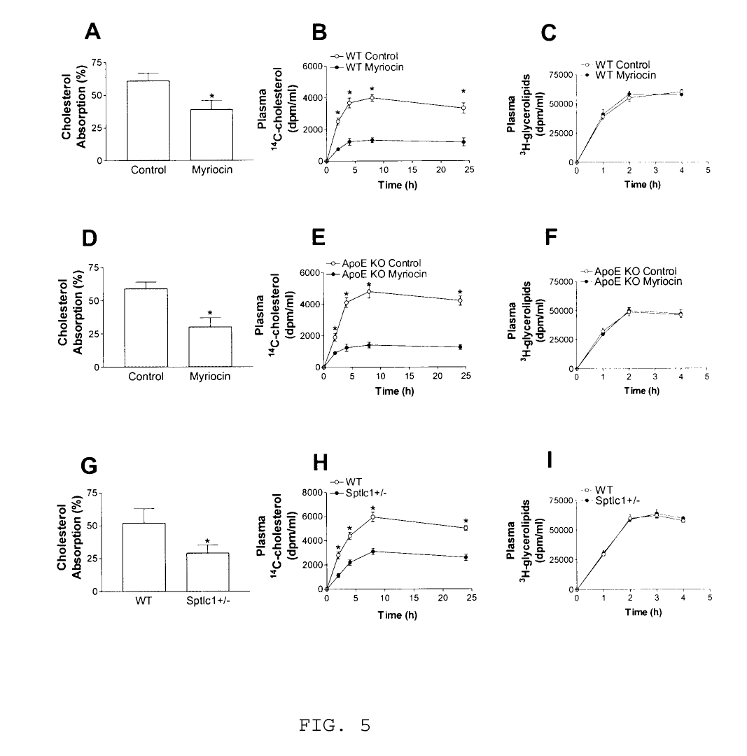

SPT activity makes an important contribution to the cell membrane structure. The interaction of SM and cholesterol drives the formation of plasma membrane rafts (11). As much as 65% of all cellular SM is found in such rafts (13). Using in situ lysenin treatment, it was observed by the present inventors for the first time that myriocin treatment or Sptlc1 gene deficiency decreases SM levels in the apical surface of the enterocytes (FIG. 5). Since lysenin recognizes SM only when it forms aggregates or domains, (24) the data from the present experiments suggest that SPT activity is responsible for the level of apical membrane SM. This in turn modulates the formation or maintenance of microdomains in the membranes, influences the molecules (such as NPC1L1 and ABCG5/G8) that are located in such microdomains, (2, 17) and, thus, influences cholesterol absorption.

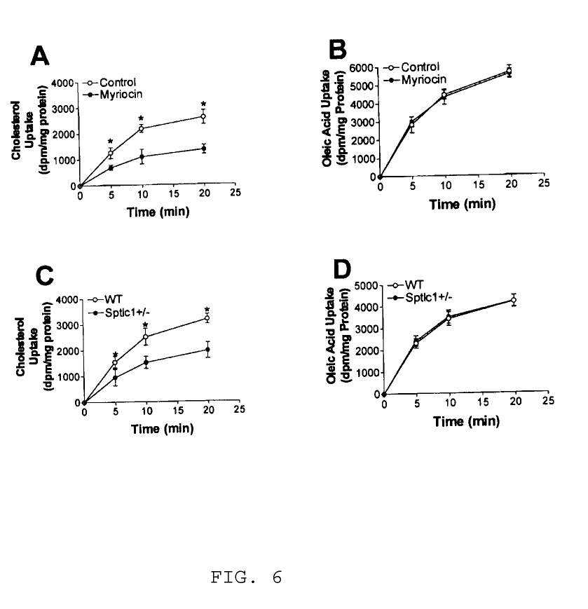

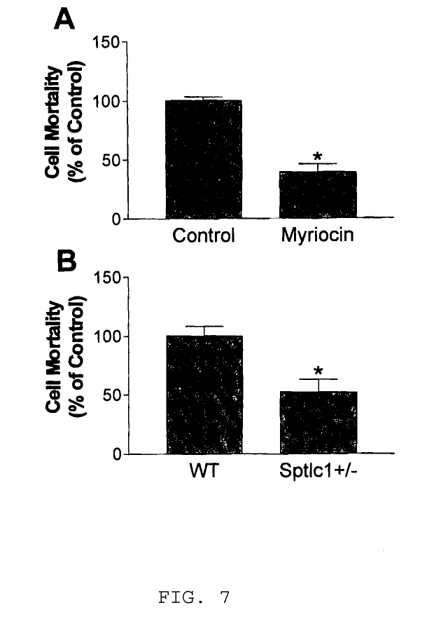

The present invention is directed to the discovery that SPT specifically regulates cholesterol uptake without affecting that of oleic acid. The SPT deficiency decreased NPC1L1 (FIG. 6) protein but not mRNA levels (FIGS. 7A-C), suggesting that regulation is translational or posttranslational. Moreover, since mouse liver has no detectable NPC1L1, the observed phenotype (decreased cholesterol in apoE KO mouse plasma) (Table 1), is small intestine-dependent. NPC1L1 has recently been shown to reside mainly in the intracellular organelles, and is transported to apical membrane when cells are deprived of micellar cholesterol.(2) The cholesterol-NPC1L1 interaction and structural assembly of NPC1L1 may influence the kinetics of net cholesterol movement across the cell membranes of the enterocyte. It is may therefore be important to investigate how structural protein integrity or assembly at the cell membrane level is maintained during the intestinal absorption of cholesterol. It is possible that SPT deficiency affects the SM composition of cellular organelles, such as plasma membrane and the endoplasmic reticulum, and alters intracellular transport of NPC1L1, resulting in reduced NPC1L1 proteins and assimilation of cholesterol by the enterocytes.

As shown by the present inventors, SPT deficiency increases small intestine ABCG5 protein levels. Human sitosterolemia is caused by mutations in either ABCG5 or ABCG8.(28) Intestinal absorption of cholesterol in patients with sitosterolemia is increased by about 30% and intestinal absorption of sitosterol is increased by about 800%, as measured by plasma dual-isotope labeling methods.(29) These increments in humans are consistent with mouse studies in ABCG5/G8 KO animals.(30) Over expression of ABCG5 and ABCG8 reduces fractional absorption of dietary cholesterol.(31) Immunohistochemical analyses show that ABCG5 and ABCG8 proteins are apically localized in the small intestine.(17) It is possible that SPT deficiency affects SM composition on the apical surface of the enterocytes and increases ABCG5/G8 proteins, resulting in more cholesterol being pumped back into the lumen of the intestine, and less of it being absorbed. However, the possibility that the regulation of ABCG5/G8 is transcriptional in the small intestine is ruled out, since ABCG5/G8 mRNA levels are increased there but not in the liver (FIG. 7).

SPT deficiency also decreases ABCA1 levels in the small intestine. The present inventors have shown that apoAI and ABCA1 play a role in intestinal cholesterol secretion, and this process also makes a contribution to cholesterol absorption.(5, 6) ABCA1 resides in the basal membranes of the enterocytes.(32) ABCA1-dependent cholesterol export involves an initial interaction of apoAI with lipid raft membrane domains.(33) It is conceivable that SPT deficiency not only decreases SM levels on the apical surface of the enterocytes (FIG. 5), but also decreases SM levels in the basal membranes where ABCA1 is located. The down regulation of ABCA1 might also make a contribution to decreased cholesterol absorption in SPT-deficient mice. Again, it is possible to rule out that the regulation of ABCA1 is transcriptional in the small intestine, since ABCA1 mRNA levels are increased there (FIGS. 7A-C).

The possible involvement of sphingolipids, other than SM, in the regulation of NPC1L1, ABCG5/G8, and ABCA1, cannot be excluded since SPT is the key enzyme for biosynthesis of all the sphingolipids, including ceramide and sphingosine-1-phosphate. (7) Indeed, cholesterol efflux to apo A-I requires plasma membrane ceramide structural features.(34) FTY720, an analogue of sphingosine-1-phosphate, increases plasma cholesterol levels in apoE KO mice.(35) The detailed mechanism involved in the regulation of NPC1L1, ABCG5/G8, and ABCA1 in the SPT-deficient small intestine requires further investigation.

In summary, the present invention may use the unexpected discovery that SPT deficiency leads to less cholesterol absorption in order to screen for potential drug candidates that may act as SPT inhibitors or cholesterol absorption inhibitors. This is associated with reduced SM levels on the apical surface of the enterocytes, as well as decreased NPC1L1 and increased ABCG5 proteins in the small intestine. This may also relate to reduction of SM levels in the basal membrane of the enterocytes, and down regulation of ABCA1 in these cells. Decreased cholesterol absorption could be a mechanism contributing to the low cholesterol levels and decreased atherosclerosis found in SPT-deficient apoE KO mice. (18, 25) Thus, SPT might be an excellent candidate for therapeutic intervention, and its inhibition could be very useful in lowering plasma cholesterol levels and decreasing atherosclerosis.

The present invention is directed to a method of screening cholesterol absorption inhibitors. The method of screening cholesterol absorption inhibitors includes the steps of administering to a mammal a biologically effective amount of a candidate SPT inhibitor; and determining whether an amount at least one cholesterol absorption indicator protein in the intestine has changed after the administering step.

The administration step may be done by injection, intravenous subcutaneous intraperitoneal, or intramuscular, and other methods of administration, as are known in the art and as may be desired.

The determining may further include comparing a pre-administration level of the cholesterol absorption indicator to a post administration level of said cholesterol absorption indicator. The cholesterol absorption indicator may refer to a level of a key protein indicative of cholesterol absorption in the small intestine, including ABCG5, ABCG8, ABCA1, and/or NPC1L1. The determining step may refer to determining the level of one or more of the key factors, as descried. Further, the cholesterol absorption indicator may be an SM level, which is measurable in one or more means, as may be desired. The determining step may further include comparing the amount of cholesterol absorption indicator with a standard level.

When an individual, such as a doctor or clinician is determining whether a cholesterol reduction indicator has changed, the use of a standard medical text, correlation program, or comparative results based on a previous test may be used. The acceptable or traditional levels of these proteins for lower or very small amounts of cholesterol absorption may be calculated or known from standards, published values, or the like. Measurement of protein levels may be done, for example, by western blot, measuring total RNA, or other methods as is known in the art.

The reduction of an NPC1L1 amount or an ABCA1 is indicative of a blocked cholesterol absorption in the small intestine. Thus cholesterol absorption is inhibited, thereby blocking any subsequent cholesterol metabolism. An increase in the amount of ABCG5 is indicative of a blocked cholesterol absorption in the small intestine. Thus cholesterol absorption is inhibited, thereby blocking any subsequent cholesterol metabolism.

The method may further include the step of measuring a spingomyelin level in the small intestine. The level may be subsequently analyzed as against medical standards found in textbooks, charts, computerized programs, and readily understood in the profession. The absence or reduction of SM in the small intestine is a factor that may be indicative that cholesterol absorption is inhibited.

Similarly, the method of screening SPT inhibitors may further include the step of determining whether the small intestine and/or small intestine apical surface has undergone a pathological change as a result of the administration of the candidate SPT inhibitor. If there are pathological changes, the structure or function of the small intestine may be detrimentally affected and the subject or mammal may be harmed or have permanent changes or loss of function to the small intestine. Instead, it is desirable that a successful SPT inhibitor candidate effect the small intestine only in its biochemistry of absorption and not create any anatomical or pathological change to the small intestine. Therefore, the method of screening candidate SPT inhibitors may further comprise the step of determining whether a biochemical or pathological change has occurred in the small intestine surface. Determining whether either of said changes may be accomplished by performing one or more tests or procedures. For example, the small intestine may be visually inspected, microscopically inspected, tested, or otherwise assayed to determine whether or nor the small intestine has undergone a pathological change, a biochemical change, or both to reduce cholesterol absorption.

The method may result in a biochemical change to the apical surface of the small intestine. This biochemical change is measurable by completing assays on one or more of the proteins previously referenced, where the change in protein levels corresponds to a lowered ability of the enterocyte surface to absorb cholesterol in the small intestine. Thus, the mammal has a resultingly reduced ability to absorb cholesterol from consumables. The cholesterol in the circulation and in the plasma levels is thereby reduced.

Generally, the use of serine palmitoyltransferase (SPT) inhibitor may be employed for causing a biochemical change in the surface of an apical protein on a portion of small intestine. This change may result in the lowering of cholesterol absorption into a subject body, which may be used to treat various cholesterol related diagnoses, including for example, dyslipidemia and atherosclerosis, or related disorder. The methods of the present invention may thus be used in order to effectively screen drugs in an quick and efficient animal cholesterol absorption test, SM level test, key protein test (including testing for levels of NPC1L1, ABCA1, ABCG5, and/or ABCG8), and similar screening tests.

Further, as SPT ablation decreased cholesterol but not triglyceride absorption, only lipid and not triglyceride levels are affected by the biochemical change of the surface of the intestine. Decreased absorption of cholesterol was correlated with lower levels of NPC1L1 and ABCA1, and higher levels of ABCG5/G8, in the small intestine. Once one or more successful SPT inhibitors have been identified, the SPT inhibitor may be employed in a method of reducing cholesterol absorption in an organism, preferably a mammal. The mammal can be one or more common laboratory experimental species, including, hamsters, guinea pigs, mice, rats, rabbits, and the like. Similarly, the mammal may be a primate, including for example a chimpanzee or a monkey. Also, the mammal may be a human subject.

The method of biochemically reducing cholesterol absorption may include administering to a mammal a biologically effective amount of an SPT inhibitor that reduces at least one or an NPC1L1 level and an ABCA1 level, and increases an ABCG5 and/or an APCG8 level. The method of biochemically reducing cholesterol absorption may include reiterating or repeating a biologically effective dosage or treatment in a pattern, cycle, or treatment plan. Further, the progress of the method in reducing cholesterol may be monitored by one or more of the previously discussed steps, including measuring, determining various pre and post administration levels of proteins, and comparing the results thereof.

Once an SPT inhibitor is identified, it may be employed in a method of decreasing cholesterol absorption in a human. The method may include administering to a human a biologically effective amount of an SPT inhibitor which decreases an NPC1L1 protein level, increases an ABCG5 protein level or increases an ABCG8 protein level in an apical surface of a small intestine; decrease ABCA1 protein level in the basal membrane of a small intestine.

The sphingomyelin decreasing drug candidates that are identified have numerous utilities. For example, they may be effective in inhibiting cholesterol absorption and/or reducing inflammation. Similarly, they may be employed against one or more diagnoses related to these applications, either individually or on combination with one or more known compounds or treatments. The inhibitor may be in a pharmaceutical formulation, as previously described.

For example, the candidate drug may increase or decrease one or more protein levels in the apical surface of the enterocyte of the small intestine. Various methods of determining an increase or decease in protein level may be used, as desired. Particularly, the candidate drug may effect an NPC1L1 level, an ABCA1 level, or an ABCG8 level, a combination of two protein levels, or all three protein levels. The levels may increase or decrease in a measurable and/or observable fashion or effect.

Similarly, the knowledge generated with the present invention may include a method of decreasing cholesterol absorption in a human, of treating diseases including atherosclerosis and dyslipidemia. The various methods may be employed to create a biochemical change on an enterocyte apical surface of a small intestine, wherein protein levels of proteins, including but not limited to the key factors of NPC1L1, ABCA1, ABCG5, and ABCG8 exhibiting a change, while the pathology of the intestine exhibits no observable change. Thus this resulting change is biochemical, not morphological or pathological. Similarly, a method for decreasing cholesterol absorption in a subject may comprise inhibiting a serine palmityolCoA transferase (SPT) level in an enterocyte apical surface of a portion of small intestine of the subject.

### EXAMPLES, EXPERIMENTAL, AND RESULTS

### Methods

Materials: [9,10(N)-³H]triolein was from NEN Life Science Products. [4-¹⁴C]cholesterol and [9,10(N)-³H]oleic acid were from Amersham. [5,6-³H]sitostanol was from American Radiolabeled Chemicals. Oleic acid (OA) was from Sigma. Dulbecco's modified Eagle's medium (DMEM) was from Invitrogen.

Mice and diet: Male C57BL/6J and apoE KO (Apolipoprotien E knockout) mice with a C57BL/6J background were obtained from the Jackson Laboratory (Bar Harbor, Me.). Sptlc1 heterozygous KO mice with a C57BL/6 background were created and bred in the inventors' laboratory. Myriocin was mixed with a chow diet. Eight- to 12-week-old WT or apoE KO (n=6) mice received myriocin 0.3 mg kg⁻¹·d⁻¹ for 12 weeks. The myriocin dose was chosen from a previous dose-dependent experiment with apoE-KO mice. Controls consisted of WT or apoE KO mice fed a chow diet (n=6). Sptlc1 heterozygous KO (Sptlc1^{+/-})(n=6) and WT mice were also fed a chow diet.

Cholesterol absorption studies: A classical fecal dual-isotope ratio method was used for the cholesterol absorption study. Briefly, a mixture of [¹⁴C]-labeled (0.1 microCi) and unlabeled cholesterol (0.5 mg) and [³H]sitostanol (0.2 microCi) in 15 microl of olive oil was fed to mice (10-12 weeks old). Feces were collected for 24 hours. The cholesterol absorption ratio was calculated as: % absorption={1−[fecal(¹⁴C/³H)]/administered(¹⁴C/³H)}×100. In some cases, mice were sacrificed, plasma collected, and radioactivity counted. Small intestines (from duodenum to ileum) were washed and cut into 2 cm segments. Each of these, as well as a part of the liver, were digested and radioactivity counted individually.

Cholesterol uptake by primary enterocyte: The enterocyte cholesterol uptake study was carried out as disclosed in: Iqbal J, Hussain M M. Evidence for multiple complementary pathways for efficient cholesterol absorption in mice. J Lipid Res. 2005; 46:1491-1501, which is incorporated herein by reference in its entirety.

Tissue SPT activity assay: Mouse small intestine (0.2 g) was homogenized in 0.5 ml of 50 mM Tris-HCl, pH 7.4, 5 mM EDTA, and 250 mM sucrose. SPT activity in the homogenates was measured with [¹⁴C]-serine and palmitoyl-coenzyme A for substrates, as previously described (22), which is incorporated herein by reference in its entirety.

Real-time PCR examined genes' expression: Mice were sacrificed by cervical dislocation. Jejunum was dissected, and total RNA extracted with Trizol (Invitrogen). cDNA was synthesized with an Invitrogen kit. Polymerase chain reaction (PCR) was performed on a total volume of 20 microl with the sybergreen kit from Applied Biosystems, 18S being used as an internal control. The amplification program consisted of activation at 95 degrees C. for 10 minutes, followed by 40 amplification cycles: 95 degrees C. for 15 seconds, 60 degrees C. for 1 minute. Each sample was triplicated. The genes' relative expression was expressed as mean±SD. The primers are located in the below table which lists the name, sequence, and sequence identification number.

| | | |
|---|---|---|
| SEQ ID NO. | Name | Sequence |

| | | |
|---|---|---|
| SEQ ID NO: 1 | Mouse NPC1L1 primer forward | ATCCTCATCCTGGGCTTTGC |
| SEQ ID NO: 2 | Mouse NPC1L1 primer reverse | GCAAGGTGATCAGGAGGTTGA |
| SEQ ID NO: 3 | Mouse ABCG5 primer forward | GCAGGGACCAGTTCCAAGACT |
| SEQ ID NO: 4 | Mouse ABCG5 primer reverse | ACGTCTCGCGCACAGTGA |
| SEQ ID NO: 5 | Mouse ABCG8 primer forward | AAAGTGAGGAGTGGACAGATGCT |
| SEQ ID NO: 6 | Mouse ABCG8 primer reverse | TGCCTGTGATCACGTCGAGTAG |
| SEQ ID NO: 7 | Mouse ABCA1 primer forward | TTGGCGCTCAACTTTTACGAA |
| SEQ ID NO: 8 | Mouse ABCA1 primer reverse | GAGCGAATGTCCTTCCCCA |
| SEQ ID NO: 9 | 18S rRNA, forward | AGTCCCTGCCCTTTGTACACA |
| SEQ ID NO: 10 | 18S rRNA, reverse | GATCCGAGGGCCTCA CTAAAC |

Preparation and Western blot analysis of small intestine homogenates: A total of 50-100 mg of small intestine sample was homogenized and lysed proteins were immunoblotted, as previously described, (3) with a polyclonal rabbit antihuman NPC1L1 antiserum 69B, a polyclonal rabbit antimouse ABCG5 antiserum, a polyclonal antimouse ABCA1 antibody, and a monoclonal mouse anti-(beta)β-actin antibody.

In situ lysenin treatment and cell mortality measurement: Overnight-fasted mice were anesthetized and small intestines were isolated from WT animals with or without myriocin treatment, as well as sptlc1 KO and control mice. Contents of the intestinal lumen were removed and washed with buffer containing 117 mM NaCl, 5.4 mM KCl, 0.96 mM NaH₂PO₄, 26.19 mM NaHCO₃ and 5.5 mM glucose (pH 7.4). Intestines were turned inside-out and cut into 0.5 cm segments from jejunum. These segments were then bathed in 0.5 ml of oxygenated DMEM containing 5% glutamine with lysenin (5 microg/ml), or without it as a control, at 37 degrees C. for 30 minutes. WST-1 cell proliferation reagent (50 micro 1) was added to monitor cell mortality. After continuous incubation at 37 degrees C. for 15 minutes, the solution was transferred to an Eppendorf tube and spun (12,000 rpm) to pellet cell debris. Supernatant was then measured OD at 450 nm, a reading for viable cells (WST-1 reagent with no cell incubation being the background reading). Buffers and medium were gassed with 95% O2/5% CO2 for 20 minutes, and maintained at 37 degrees C. prior to use.

Mouse small intestine hematoxylin and eosin staining: The small intestine was dissected out and put into 4% paraformaldehyde for fixation overnight. The tissue was then sliced (10 micro m thick). Each slice was deparaffinized and stained with hematoxylin and eosin.

Statistical analysis: Data were expressed as mean±SD. Differences between groups were evaluated by Mann-Whitney U test (non-parametric test). P values less than 0.05 were considered significant.

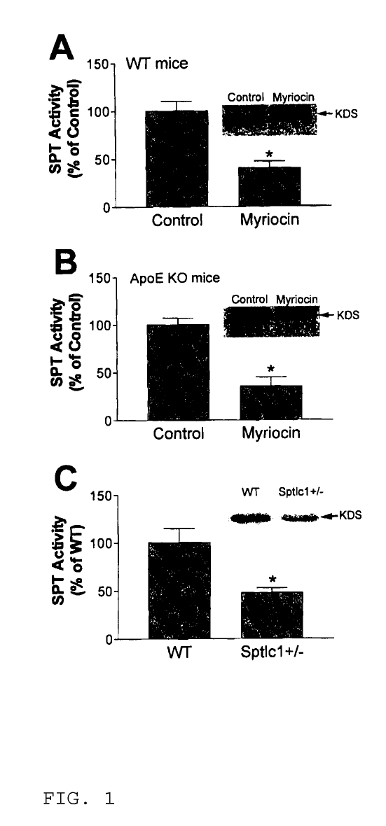

Myriocin treatment or Sptlc1 deficiency significantly decreases SPT activity without altering the epithelial structure of the small intestine. To investigate the relationship between small intestine SPT activity and cholesterol absorption, pharmacological and genetic approaches were utilized. For the first set of experiments: four groups (n=6 per group) of 12-week-old WT and apoE KO mice were used. Groups 1 and 2 were WT mice fed a chow diet with or without myriocin for 3 weeks; groups 3 and 4 were apoE KO mice fed a chow diet with or without myriocin for 3 weeks. Myriocin-treated mice had 60 to 65% less SPT activity respectively than controls in the small intestine (FIGS. 1A and B). For the second set of experiments: Sptlc1 heterozygous KO (Sptlc1^{+/-}) mice (n=6) and WT controls (n=6) were utilized. Sptlc1^{+/-} mice had 52% less SPT activity than controls in the small intestine (FIG. 1C). These studies indicated that myriocin treatment and Sptlc1 deficiency reduced small intestinal SPT activity.

As shown in Table 1, myriocin treatment significantly decreased plasma SM (48%, P<0.001) and cholesterol (37%, P<0.01) in apoE KO mice, compared with controls. On the other hand, myriocin treatment caused no significant effect on plasma lipids, including SM, PC, and cholesterol levels, in WT mice. As reported previously in (25), which is incorporated by reference herein, Sptlc1^{+/-} and WT mice had identical plasma lipid levels (Table 1).

TABLE 1
Mouse plasma lipid measurement.

| | SM | PC | Chol | TG |
|---|---|---|---|---|
| | mg/dl | | | |
| WT mice | | | | |
| Control | 25 ± 5 | 139 ± 19 | 101 ± 17 | 79 ± 12 |
| Myriocin | 22 ± 2 | 155 ± 31 | 95 ± 12 | 71 ± 15 |
| ApoEKO mice | | | | |
| Control | 75 ± 9 | 289 ± 25 | 556 ± 39 | 105 ± 19 |
| Myriocin | 39 ± 4* | 334 ± 49 | 350 ± 42* | 89 ± 13 |
| Mice | | | | |
| WT | 22 ± 3 | 127 ± 24 | 91 ± 11 | 89 ± 16 |
| Sptlc1^{+/-} | 19 ± 5 | 138 ± 33 | 99 ± 8 | 92 ± 10 |

Value, mean ± SD.
*P < 0.01,
N = 6.
SM = sphingomyelin;
PC = phosphatidylcholine;
Chol = cholesterol;
TG = triglycerol.

To investigate whether myriocin treatment or Sptlc1^{+/-} had any impact on small intestine morphology, intestinal sections were stained with hematoxylin and eosin. As shown in FIGS. 2 through 4, neither myriocin treatment nor Sptlc1^{+/-} influenced the morphology of the small intestine. All specimens had intact villi, epithelia, enterocytes, goblet cells, and brush borders. Therefore, SPT deficiency does not affect gross small intestinal morphology.

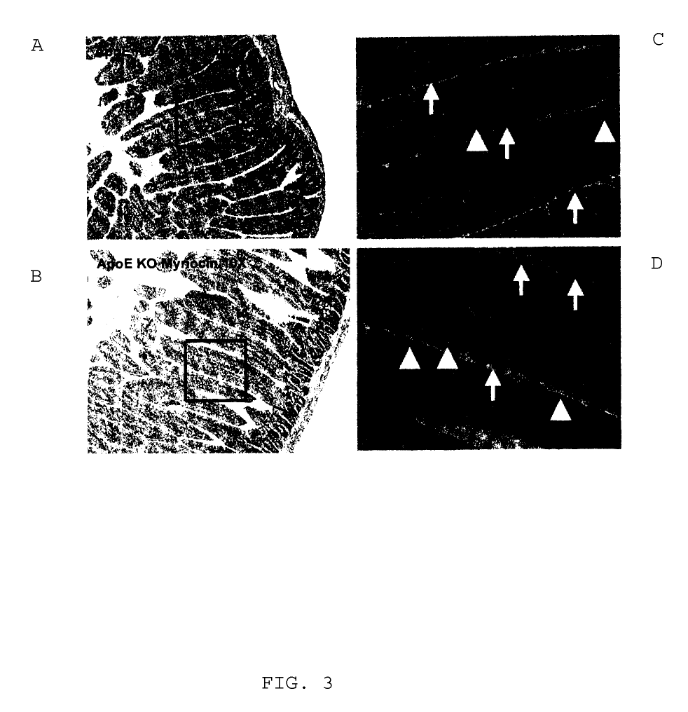

Myriocin treatment or Sptlc1 deficiency significantly decreases cholesterol but not triacylglycerol absorption. The observed decrease of plasma cholesterol levels in apoE KO mice after myriocin treatment (Table 1) could be due to a defect in cholesterol absorption. To explore the relationship between SPT deficiency and cholesterol absorption, studies after a single gavage were completed, using the conventional fecal dual-isotope ([¹⁴C]-cholesterol/[³H]sitostanol) ratio method.^{6,20,21} As shown in FIG. 5, there was a significant reduction in cholesterol absorption in myriocin-treated WT (36%, P<0.01) (FIG. 5A), myriocin-treated apoE KO (50%, P<0.001) (FIG. 5D), and Sptlc1^{+/-} mice (43%, P<0.001) (FIG. 3G), compared with controls.

Blood [¹⁴C]-cholesterol levels within 24 hours after a single gavage were monitored, and it was found that myriocin-treated WT (FIG. 3B), myriocin-treated apoE KO (FIG. 3E), and Sptlc1^{+/-} mice (FIG. 3H) had significantly less [¹⁴C]-cholesterol in the circulation than controls. This confirmed that there was defective cholesterol absorption in these animals.

To investigate whether the effect of SPT deficiency was specific to cholesterol, experimental mice were fed with 0.1 microCi [³H]triolein instead of [¹⁴C]cholesterol, and blood was collected at different time points within 24 hours. No significant changes in the [³H]triolein-derived counts in the plasma between SPT-deficient (myriocin treated or Sptlc1^{+/-}) animals and controls were observed (FIGS. 3C, F, and I), indicating that SPT deficiency has no effect on triglyceride absorption.

Next, the amounts of [¹⁴C]cholesterol present in the intestine and those transported to plasma, liver, and bile in 24 hours were measured after a single bolus of radiolabeled cholesterol. Myriocin-treated WT and apoE KO, as well as Sptlc1^{+/-} small intestines, plasma, livers, and bile contained significantly less [¹⁴C]cholesterol than controls (Table 2). Lower counts in plasma, liver, and bile indicated that SPT deficiency in mice caused less efficiency in cholesterol absorption.

TABLE 2
Absorption of [¹⁴C]cholesterol after a single gavage

| | Intestine | Liver | Bile | Plasma |
|---|---|---|---|---|
| | (dpm/g) | (dpm/g) | (dpm/ml) | (dpm/ml) |
| WT mice | | | | |
| Control | 10103 ± 792 | 8111 ± 1038 | 5092 ± 661 | 3901 ± 674 |
| Myriocin | 5237 ± 478* | 4131 ± 575* | 2108 ± 394* | 1734 ± 390* |
| ApoE KO | | | | |
| mice | | | | |
| Control | 9814 ± 1184 | 7471 ± 781 | 4478 ± 742 | 4098 ± 615 |
| Myriocin | 4158 ± 499* | 3337 ± 649* | 2284 ± 521* | 1350 ± 142* |
| Mice | | | | |
| WT | 11122 ± 1335 | 9927 ± 1249 | 5687 ± 577 | 5891 ± 711 |
| Sptlc1^{+/-} | 6945 ± 816* | 4492 ± 398* | 2652 ± 662* | 2992 + 606* |

Mice were fed with either 0.1 microCi of [¹⁴C]cholesterol and 1 microCi of [³H]sitostanol together with 0.5 mg of unlabeled cholesterol in 15 microl of olive oil. After 24 hours, plasma, small intestine, liver, and bile were collected and used for radioactivity measurements.
Values are Mean ± SD,
n = 5.
* P< 0.01.

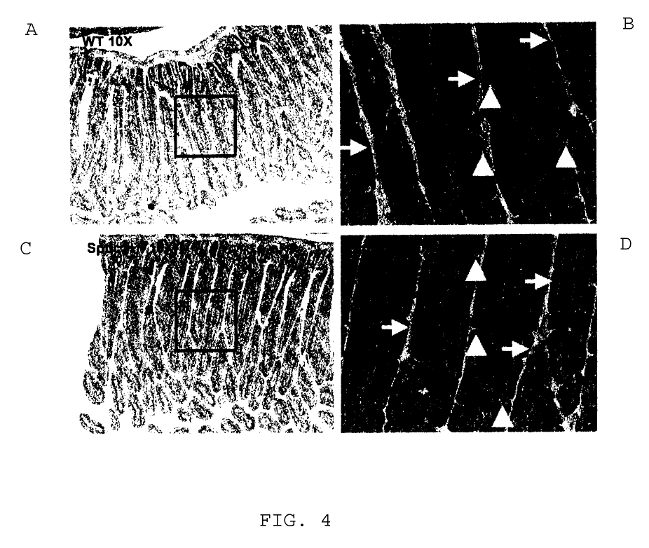

For further confirmation of the above in vivo observations, enterocytes were isolated from myriocin-treated or Sptlc1^{+/-} mice, as well as controls, incubated with radiolabeled cholesterol for varying times, and the cellular radioactivity was determined. It was determined that myriocin-treated or Sptlc1-deficient enterocytes took up significantly less radioactivity than controls, indicating a defect in cholesterol uptake (FIGS. 4A and C). Experiments were performed to study the uptake of [³H]oleic acid. No significant changes were observed (FIGS. 4B and D). These data indicate that myriocin treatment or Sptlc1 deficiency specifically decreases cholesterol uptake by the enterocytes.

Myriocin treatment or Sptlc1 deficiency significantly decreases small intestine apical membrane SM levels, decreases NPC1L1 and increases ABCG5/G8 protein levels. The mechanisms responsible for decreased cholesterol absorption in myriocin-treated or Sptlc1^{+/-} mice were explored. First, lipid levels were measured in enterocytes isolated from myriocin-treated and Sptlc1^{+/-} mice, as well as controls. It was found that myriocin-treatment significantly decreases SM levels in enterocytes, but has no effect on cellular cholesterol, phosphatidylcholine, or triglyceride levels (Table 3). It was found that Sptlc1^{+/-} and WT mice have same lipid levels (Table 3).

TABLE 3
Mouse enterocyte lipid measurement.

| | SM | PC | Chol | TG |
|---|---|---|---|---|
| | microg/mg protein | | | |
| WT mice | | | | |
| Control | 10.5 ± 0.5 | 30.6 ± 0.7 | 15.0 ± 1.1 | 11.3 ± 1.7 |
| Myriocin | 6.2 ± 0.9* | 27.9 ± 2.3 | 16.2 ± 1.2 | 11.1 ± 1.9 |
| Mice | | | | |
| WT | 9.8 ± 1.1 | 32.1 ± 1.8 | 16.0 ± 0.4 | 12.0 ± 0.4 |
| Sptlc1^{+/-} | 9.3 ± 1.4 | 30.4 ± 1.6 | 15.7 ± 0.2 | 11.7 ± 0.2 |

Value, mean ± SD.
*P < 0.01,
N = 5-7.
SM = sphingomyelin;
PC = phosphatidylcholine;
Chol = cholesterol;
TG = triglycerol.

These results suggest that cellular lipid levels may have little or no effect on the observed phenotype, i.e. decreasing cholesterol absorption. Second, lysenin, a SM-specific cytotoxin, was used to measure apical membrane SM levels, since lysenin can recognize SM only when it forms aggregates or microdomains in the plasma membranes. Relevant discussion can be found in reference 24, which is incorporated herein by reference in its entirety. An in situ lysenin assay was performed: intestines were turned inside-out and cut into 0.5 cm segments from jejunum, and these were incubated with 5 micro g/ml lysenin. Cell viability in tissue segments was measured by adding WST-1 cell proliferation reagent. As indicated in FIG. 7, intestinal segments from myriocin-treated or Sptlc1^{+/-} mice showed significantly less sensitivity to lysenin-mediated cytolysis than controls, indicating a decrease of SM levels in the apical membranes.

Next, Western blot measurements of NPC1L1 and ABCG5/G8 were completed, where the measurement were located in apical membranes of the enterocytes. (2, 17) it was found that myriocin treatment significantly decreased NPC1L1 and increased ABCG5 protein mass in WT and apoE KO mice, compared with nontreated animals (FIG. 6A). Moreover, small intestine from Sptlc1^{+/-} mice contained significantly less NPC1L1 and more ABCG5 than that from WT mice (FIG. 6B). These results suggested that a decrease in apical membrane SM levels could decrease those of NPC1L1 and increase those of ABCG5/G8, thus diminishing cholesterol absorption. Also, ABCA1 was measured, which is located in the basal membranes of the enterocytes and is involved in cholesterol secretion. (6) ABCA1 was decreased in SPT-deficient small intestines, compared with controls (FIGS. 6A and B), suggesting that the decrease in ABCA1 might also contribute to lower cholesterol absorption in these mice.

For further elucidation of the possible mechanisms for cholesterol absorption reduction in the SPT-deficient small intestine, NPC1L1, ABCG5/G8, and ABCA1 mRNA levels in myriocin-treated WT, apoE KO, and Sptlc1^{+/-} mice, as well as in controls were measured. In the small intestine, it was found that myriocin treatment or Sptlc1 gene deficiency significantly decreased ABCA1 mRNA levels (in WT mice, 48%, P<0.01; in apoE KO mice, 39%, P<0.01; and in Sptlc1^{+/-} mice, 51%, P<0.01), compared with controls. It significantly increased ABCG5 and ABCG8 mRNA levels (in WT mice, 52 and 46%, P<0.01; in apoE KO mice, 69 and 63%, P<0.01; in Sptlc1^{+/-} mice, 49 and 53%, P<0.01), respectively, compared with controls. No significant changes were observed in NPC1L1 mRNA levels (FIG. 7A). Moreover, intestinal MTP activity was not influenced by SPT deficiency (data not shown). Also, the liver mRNA levels were measured to find that myriocin treatment or Sptlc1 deficiency has no influence on ABCG5/G8 mRNA levels (FIG. 7B). However, myriocin treatment but not Sptlc1 deficiency significantly decreased ABCA1 mRNA levels (in WT mice, 20%, P<0.05; in apoE KO mice, 18%, P<0.05; and in Sptlc1^{+/-} mice, 16%, P=0.07). NPC1L1 mRNA in WT or Sptlc1^{+/-} livers were not detected (data not shown).

The protocols described herein for carrying out the claimed methods are well known in the art, and are generally described in these references.
1. Gylling H, Miettinen T A. The effect of cholesterol absorption inhibition on low density lipoprotein cholesterol level. Atherosclerosis. 1995; 117:305-308.
2. Garcia-Calvo M, Lisnock J, Bull H G, Hawes B E, Burnett D A, Braun M P, Crona J H, Davis H R Jr, Dean D C, Detmers P A, Graziano M P, Hughes M, Macintyre D E, Ogawa A, O'neill K A, Iyer S P, Shevell D E, Smith M M, Tang Y S, Makarewicz A M, Ujjainwalla F, Altmann S W, Chapman K T, Thornberry N A et al. The target of ezetimibe is Niemann-Pick C1-Like 1 (NPC1L1). Proc Natl Acad Sci USA. 2005; 102:8132-8137.
3. Yu L, Bharadwaj S, Brown J M, Ma Y, Du W, Davis M A, Michaely P, Liu P, Willingham M C, Rudel L L. Cholesterol-regulated translocation of NPC1L1 to the cell surface facilitates free cholesterol uptake. J Biol Chem. 2006; 281:6616-6624.
4. Yu L, York J, von Bergmann K, Lutjohann D, Cohen J C, Hobbs H H. Stimulation of cholesterol excretion by the liver X receptor agonist requires ATP-binding cassette transporters G5 and G8. J Biol Chem. 2003; 278:15565-15570.
5. Hussain M M, Fatma S, Pan X, Iqbal J. Intestinal lipoprotein assembly. Curr Opin Lipidol. 2005; 16:281-285.
6. Iqbal J, Hussain M M. Evidence for multiple complementary pathways for efficient cholesterol absorption in mice. J Lipid Res. 2005; 46:1491-1501.
7. Merrill A H, Jones D D. An update of the enzymology and regulation sphingomyelin metabolism. Biochim Biophysi Acta. 1990; 1044:1-12.
8. Weiss B, and Stoffel W, Human and murine serine-palmitoyl-CoA transferase—cloning, expression and characterization of the key enzyme in sphingolipid synthesis. Eur J Biochem. 1997; 249:239-247.
9. Hanada K, Hara T, Nishijima M, Kuge O, Dickson R C, Nagiec M M, A mammalian homolog of the yeast LCB1 encodes a component of serine palmitoyltransferase, the enzyme catalyzing the first step in sphingolipid synthesis. J Biol Chem. 1997; 272:32108-32114.
10. Hornemann T, Richard S, Rütti M F, Wei Yu, von Eckardstein A. Mammalian serine-palmitoyltransferase—cloning and initial characterisation of a new subunit. J Biol Chem. 2006; 281:37275-37281.
11. Simons K, Ikonen E. Functional rafts in cell membranes. Nature. 1997; 387:569-572.
12. Simons K, Ikonen E. How cells handle cholesterol. Science. 2000; 290:1721-1726.
13. Li Z, Hailemariam T K, Zhou H, Li Y, Duckworth D C, Peake D A, Zhang Y, Kuo M S, Cao G, Jiang X C. Inhibition of sphingomyelin synthase (SMS) affects intracellular sphingomyelin accumulation and plasma membrane lipid organization. Biochim Biophys Acta. 2007; 1771:1186-1194.
14. Christiansen K, Carlsen J. Microvillus membrane vesicles from pig small intestine. Purity and lipid composition. Biochim Biophys Acta. 1981; 647:188-195.
15. Brown D A, Rose J K. Sorting of GPI-anchored proteins to glycolipid-enriched membrane subdomains during transport to the apical cell surface. Cell. 1992; 68:533-544.
16. Field F J, Born E, Murthy S, Mathur S N. Caveolin is present in intestinal cells: role in cholesterol trafficking? J Lipid Res. 1998; 39:1938-1950.
17. Graf G A, Yu L, Li W P, Gerard R, Tuma P L, Cohen J C, Hobbs H H. ABCG5 and ABCG8 are obligate heterodimers for protein trafficking and biliary cholesterol excretion. J Biol Chem. 2003; 278:48275-48282.
18. Park T S, Panek R L, Mueller S B, Hanselman J C, Rosebury W S, Robertson A W, Kindt E K, Homan R, Karathanasis S K, Rekhter M D. Inhibition of sphingomyelin synthesis reduces atherogenesis in apolipoprotein E-knockout mice. Circulation. 2004; 110:3465-3471.
19. Hojjati M R, Li Z, Jiang X C Serine palmitoyl-CoA transferase (SPT) deficiency and sphingolipid levels in mice. Biochim Biophys Acta. 2005; 1737:44-51.
20. Sehayek E, Ono J G, Shefer S, Nguyen L B, Wang N, Batta A K, Salen G, Smith J D, Tall A R, Breslow J L. Biliary cholesterol excretion: a novel mechanism that regulates dietary cholesterol absorption. Proc Natl Acad Sci USA. 1998; 95:10194-10199.
21. Wang D Q, Carey M C. Measurement of intestinal cholesterol absorption by plasma and fecal dual-isotope ratio, mass balance, and lymph fistula methods in the mouse: an analysis of direct versus indirect methodologies. J Lipid Res. 2003; 44:1042-1059.
22. Gable K, Slife H, Bacikova D, Monaghan E, Dunn T M. Tsc3p is an 80-amino acid protein associated with serine palmitoyltransferase and required for optimal enzyme activity. J Biol Chem. 2000; 275:7597-7603.
23. Yu L., Cao G., Repa J., Stangl H. Sterol regulation of scavenger receptor class B type I in macrophages. J Lipid Res. 2004; 45:889-899.
24. Ishitsuka R, Kobayashi T Cholesterol and lipid/protein ratio control the oligomerization of a sphingomyelin-specific toxin, lysenin. Biochemistry. 2007; 46:1495-1502.
25. Hojjati M R, Li Z, Zhou H, Tang S, Huan C, Ooi E, Lu S, Jiang X C. Effect of myriocin on plasma sphingolipid metabolism and atherosclerosis in apoE-deficient mice. J Biol Chem. 2005; 280:10284-10289.
26. Weisgraber K H, Innerarity T L, Rall S C Jr, Mahley R W Receptor interactions controlling lipoprotein metabolism. Can J Biochem Cell Biol. 1985; 63:898-905.
27. Plump A S, Smith J D, Hayek T, Aalto-Setälä K, Walsh A, Verstuyft J G, Rubin E M, Breslow J L Severe hypercholesterolemia and atherosclerosis in apolipoprotein E-deficient mice created by homologous recombination in ES cells. Cell. 1992; 71:343-353.
28. Berge K E, Tian H, Graf G A, Yu L, Grishin N V, Schultz J, Kwiterovich P, Shan B, Barnes R, Hobbs H H. Accumulation of dietary cholesterol in sitosterolemia caused by mutations in adjacent ABC transporters. Science. 2000; 290:1771-1775.
29. Salen G, Tint G S, Shefer S, Shore V, Nguyen L. Increased sitosterol absorption is offset by rapid elimination to prevent accumulation in heterozygotes with sitosterolemia. Arterioscler Thromb 1992; 12:563-568.
30. Wang H H, Patel S B, Carey M C, Wang D Q. Quantifying anomalous intestinal sterol uptake, lymphatic transport, and biliary secretion in Abcg8(−/−) mice. Hepatology. 2007; 45:998-1006.
31. Yu L, Li-Hawkins J, Hammer R E, Berge K E, Horton J D, Cohen J C, Hobbs H. Overexpression of ABCG5 and ABCG8 promotes biliary cholesterol secretion and reduces fractional absorption of dietary cholesterol. J Clin Invest. 2002; 110:671-680.
32. Lawn R M, Wade D P, Garvin M R, Wang X, Schwartz K, Porter J G, Seilhamer J J, Vaughan A M, Oram J F. The Tangier disease gene product ABC1 controls the cellular apolipoprotein-mediated lipid removal pathway. J Clin Invest. 1999; 104: R25-R31.
33. Gaus K, Kritharides L, Schmitz G, Boettcher A, Drobnik W, Langmann T, Quinn C M, Death A, Dean R T, Jessup W. Apolipoprotein A-1 interaction with plasma membrane lipid rafts controls cholesterol export from macrophages. FASEB J. 2004; 18:574-576.
34. Witting S R, Maiorano J N, Davidson W S. Ceramide enhances cholesterol efflux to apolipoprotein A-I by increasing the cell surface presence of ATP-binding cassette transporter A1. J Biol Chem. 2003; 278:40121-40127;
35. Klingenberg R, Nofer J R, Rudling M, Bea F, Blessing E, Preusch M, Grone H J, Katus H A, Hansson G K, Dengler T J. Sphingosine-1-phosphate analogue FTY720 causes lymphocyte redistribution and hypercholesterolemia in ApoE-deficient mice. Arterioscler Thromb Vasc Biol. 2007; 27:2392-2399.

Various changes and modifications can be made in the present invention. It is intended that all such changes and modifications come within the scope of the invention as set forth in the following claims.

What is claimed is:

1. A method of screening cholesterol absorption inhibitors, comprising:
administering to a mammal a biologically effective amount of a candidate SPT inhibitor;
determining whether an amount of at least one cholesterol absorption indicator protein has changed after the administering step.

2. The method of claim 1, wherein the determining step further comprises comparing a pre-administration level of said cholesterol absorption indicator to a post administration level of said cholesterol absorption indicator.

3. The method of claim 1, further wherein the determining step further comprises comparing said amount of said cholesterol absorption indicator with a standard level.

4. The method of claim 1, wherein the determining step further comprises determining the level of at least one of an NPC1L1 protein; an ABCG5 protein; and an ABCG1 protein.

5. The method of claim 4, wherein the reduction of said NPC1L1 or said ABCA1 is indicative of a blocked cholesterol absorption in the small intestine.

6. The method of claim 4, wherein the increase of said ABCG5 indicates a reduced cholesterol absorption in the small intestine.

7. The method of claim 1, further comprising the step of measuring a spingomyelin level in said small intestine of said mammal.

8. The method of claim 7, further comprising analyzing a level of said spingomyelin to determine whether said spingomyelin has been reduced which indicates a reduction in cholesterol absorption.

9. The method of claim 1, further comprising the step of determining whether said SPT inhibitor candidate changed a biochemistry on a small intestine surface wherein a biochemical change is indicative of a reduction in cholesterol absorption.

10. The method of claim 1, further comprising the step of determining whether said candidate SPT inhibitor changed a pathology of a small intestine surface.

11. A method of biochemically reducing cholesterol absorption, comprising administering to a mammal a biologically effective amount of an SPT inhibitor that reduces at least one of an NPC1L1 level and an ABCA1 level, and increases an ABCG5 level.


SEQUENCE LISTING (10 sequences)

SEQ ID NO: 1 - DNA, 20 bp, Artificial Sequence
  Mouse NPC1L1 primer forward
atcctcatcctgggctttgc

SEQ ID NO: 2 - DNA, 21 bp, Artificial Sequence
  Mouse NPC1L1 primer reverse
gcaaggtgatcaggaggttga

SEQ ID NO: 3 - DNA, 21 bp, Artificial Sequence
  Mouse ABCG5 primer forward
gcagggaccagttccaagact

SEQ ID NO: 4 - DNA, 18 bp, Artificial Sequence
  Mouse ABCG5 primer reverse
acgtctcgcgcacagtga

SEQ ID NO: 5 - DNA, 23 bp, Artificial Sequence
  Mouse ABCG8 primer forward
aaagtgaggagtggacagatgct

SEQ ID NO: 6 - DNA, 22 bp, Artificial Sequence
  Mouse ABCG8 primer reverse
tgcctgtgatcacgtcgagtag

SEQ ID NO: 7 - DNA, 21 bp, Artificial Sequence
  Mouse ABCA1 primer forward
ttggcgctcaacttttacgaa

SEQ ID NO: 8 - DNA, 19 bp, Artificial Sequence
  Mouse ABCA1 primer reverse
gagcgaatgtccttcccca

SEQ ID NO: 9 - DNA, 21 bp, Artificial Sequence
  18S rRNA, forward
agtccctgccctttgtacaca

SEQ ID NO: 10 - DNA, 21 bp, Artificial Sequence
  18S rRNA, reverse
gatccgagggcctcactaaac